(19) United States
(12) Patent Application Publication
(10) Pub. No.: US 2008/0179201 A1
(43) Pub. Date: Jul. 31, 2008

(54) CASE FOR ARTIFICIAL TEETH

(75) Inventors: Andreas Hess, Schwabisch Gmund (DE); Gerd Burchard, Waldstetten (DE); Sebastian Schnabel, Stuttgart (DE); Reiner Reichert, Taferrot (DE)

Correspondence Address: VOLPE AND KOENIG, P.C., UNITED PLAZA, SUITE 1600, 30 SOUTH 17TH STREET, PHILADELPHIA, PA, 19103, US

(73) Assignee: CANDULOR AG, Wangen (CH)

(21) Appl. No.: 12/022,222

(22) Filed: Jan. 30, 2008

(30) Foreign Application Priority Data
Jan. 30, 2007 (AT) A 150/2007

(51) Int. Cl. A61C 19/10; A61B 19/02

(52) U.S. Cl. 206/83; 206/63.5

(57) ABSTRACT

A case (1) for artificial teeth is provided, with a base part (3) for receiving the teeth and a lid (4) that can be placed onto the base part (3) and cover it and the inserted teeth are provided. The boundaries (5, 6, and/or 7, 8) of the base part (3) and the lid (4) facing each other are embodied with different heights (H1 and/or H2) adjusted to each other, with at least a large part of the rear boundary (5) of the base part (3) being embodied higher than the opposite front boundary (7) thereof.

### CROSS-REFERENCE TO RELATED APPLICATIONS

This application claims the benefit of Austrian application A150/2007, filed Jan. 30, 2007, which is incorporated by reference herein as if fully set forth.

### BACKGROUND OF THE INVENTION

1. Field of the Invention

The invention relates to a case for artificial teeth with a base part accepting the teeth and a lid that can be placed onto the base part and cover it as well as the teeth inserted therein.

2. Description of the Related Prior Art

In a known case for artificial teeth (DE102004020509 B4) a tooth strip is provided, which has a teeth receiver made from wax, on which the artificial teeth are arranged, and a lid is further arranged on said tooth strip, which together with said tooth strip encompasses a hollow space for receiving the teeth. The teeth are therefore clamped in a plane located between the tooth strip and the lid, so that a natural illustration of the position of the artificial teeth is impossible.

Further a teeth holding plate made from several small tooth plates has been known (EP1243233A1), which is provided with a carrying body or wax or adhesive, into which the teeth can be impressed. In an appropriate grid the tooth plates are placed onto a rectangular base structure so that all teeth are aligned side-by-side, however lying on their backs. A clear hood covers the teeth and the carrying body and is additionally provided with a coding. Such a construction requires a lot of space and additionally, here too, no natural positioning of the artificial teeth can be illustrated.

Further, a type of box has been known (U.S. Pat. No. 6,182,820B1) having a bottom part and a lid mounted via a hinge at a perimeter wall of a bottom part, with here, as well, the teeth being inserted in the bottom part in a lying position. Additionally a foam rubber insert is provided in the lid, which in the closed state of the box presses the inserted teeth to the base of the bottom part.

### SUMMARY OF THE INVENTION

The present invention is to provide a case of the type mentioned at the outset, by which artificial teeth can be inserted in a natural positioning and in which these teeth are very visible in a front and a top view and primarily such that they can be compared sufficiently well to a constant background without falsifying the color.

According to the invention this is attained in a case for artificial teeth having a base part for receiving the teeth and a lid that can be placed onto the base part and cover it as well as the teeth inserted therein such that the boundaries of the base part and the lid facing each other are embodied at different heights adjusted to each other, with at least a large part of the rear boundary of the base part being embodied higher than the opposite front boundary thereof.

By this measure according to the invention, in spite of the always matching base part and the lid, a row of teeth can be inserted positioned upright, and due to the fact that the rear boundary of the base part is embodied higher, the teeth can be inserted freely protruding in front of this higher boundary and approximately parallel thereto. Here, the higher reaching rear boundary not only provides a type of rear wall for the teeth to be observed but it simultaneously results in a mechanical protection for the teeth when the lid is removed.

A particular embodiment provides that the rear and perhaps also the opposite front boundary of the base part are provided with an edge extending convexly arched and the respective boundaries of the lid being embodied correspondingly with an indentation. This is particularly beneficial for the insertion of the teeth and the illustration of their position.

According to another embodiment of the invention, it is also possible for the rear and perhaps also the opposite front boundary of the base part and the lid to be embodied with differently high sections following each other in a staggered manner. Therefore, various design embodiments are possible in order to create highly visible front sides of the teeth and a type of rear wall behind the teeth. However, there is always sufficient space provided between the back of the teeth and the respective boundary.

In the context with the different heights of the front and the rear boundary it is also advantageous when the lateral boundaries of the base part facing each other and the lid are provided with a diagonal, perhaps arc-shaped progressing edge adjusted to the differently high rear and front boundaries of the base part and the lid. Therefore a good continuous circumferential closure of the case is possible, which is an essential advantage for the transportation and storage.

In order to allow an appropriate securing of the closed case it is suggested to arrange bars protruding at the freely projecting interior and/or the exterior sides of the boundaries and perhaps also at the lateral boundaries of the base part and/or the lid for a mutually form-fitting engagement of the base part and the lid. This way an appropriate safety from displacement is ensured with regard to the horizontal direction.

Another variant provides for the protruding bars to be provided only at some sections of the perimeter of the boundaries, preferably only in the corner sections at the transfer of the rear and the front boundaries to the lateral boundaries. This way a facilitated placement of the lid can occur.

Further, a construction is possible in which the base part and the lid engage each other similar to a tongue-and-groove connection. Due to the fact that the case generally is embodied as a plastic part such an embodiment can also be easily produced and the opening and closing of the case can be easily performed.

Further it is suggested that in the base part a material is provided to impress artificial teeth, which adjusted to the arc-shaped or staggered edge is perhaps embodied respectively elevated in the central section in reference to the length of the base part. Therefore, the position of the artificial teeth can be determined in a simple manner. However, another possibility allows the teeth to be arranged inserted in the middle of the case such that they protrude slightly further upwards so that the teeth shown in the case are aligned in an upwardly arched row.

In one particular embodiment it is provided for the base part and the lid are embodied essentially in a rectangular fashion, seen in the horizontal cross-section. However, it is also possible that the base part and the lid, seen in a horizontal cross-section, are embodied essentially as an ellipsoid. Further, there is a design solution with the base part and the lid, seen in a horizontal cross-section, being formed by front and back boundaries extending arched in the same direction and side boundaries located therebetween, perhaps also extending plane or arched. Therefore, many embodiment variants are possible, all targeting an optimal case for artificial teeth.

Further it is suggested that at least the lid is produced from a clear or at least translucent plastic. This way it can be determined from the outside which teeth and what color of teeth are arranged in the case.

### BRIEF DESCRIPTION OF THE DRAWINGS

Using the drawings further features according to the invention and particular advantages are explained in greater detail in the following description. The drawings show:

FIG. 1 is a view of a base part of a case according to the invention with artificial teeth inserted;

FIG. 2 is a perspective view of a base part;

FIG. 3 is a perspective view of a lid;

FIG. 4 is a view of a closed case with a base part according to FIG. 2 and a lid according to FIG. 3;

FIG. 5 is a perspective view of an additional base part;

FIG. 6 is a perspective view of a lid matching the illustration in FIG. 5;

FIG. 7 is a view of a closed package with a base part according to FIG. 5 and a lid according to FIG. 6;

FIG. 8 is a perspective view of a rectangular case in a horizontal cross-section;

FIG. 9 is a top view of a base part of a case according to FIG. 8;

FIG. 10 is a perspective view of a base part similarly embodied as in FIG. 5 however showing a rectangular case in a horizontal cross-section.

FIG. 11 is a side view of a base part according to FIG. 10;

FIG. 12 is a perspective view of an arc-shaped case in a horizontal cross-section

FIG. 13 is a top view of a base part of a case according to FIG. 12;

FIG. 14 is a perspective view of a base part of a case according to FIG. 12;

FIG. 15 is a side view of the base part according to FIG. 14;

FIG. 16 is a perspective view of a kidney-shaped case in a horizontal cross-section;

FIG. 17 is a top view of the base part of a case according to FIG. 16;

FIG. 18 is a perspective view of a base part of a case according to FIG. 16;

FIG. 19 is a side view of the base part according to FIG. 18;

FIG. 20 is a perspective view of a kidney-shaped case in a horizontal cross-section, with the boundaries of the base part and the lid also extending arc-shaped;

FIG. 21 is a top view of the base part of a case according to FIG. 20;

FIG. 22 is a perspective view of a base part of a case according to FIG. 20;

FIG. 23 is a side view of the base part according to FIG. 22;

FIG. 24 is a perspective view of an ellipsoid case in a horizontal cross-section;

FIG. 25 is a top view of a base part of a case according to FIG. 24;

FIG. 26 is a perspective view of a base part of a case according to FIG. 24;

FIG. 27 is a side view of a base part according to FIG. 24;

FIG. 28 is a perspective view of a base part with different height sections at the boundary that engages the lid;

FIG. 29 is a perspective view of the matching lid for the base part of FIG. 28;

FIG. 30 is a perspective view of a closed package with a base part according to FIG. 28 and a lid according to FIG. 29;

FIG. 31 is a partial perspective view of an alternate arrangement of the base of FIG. 31;

FIG. 32 is a perspective view of a base part with bars extending from the inner boundary where the lid is engaged;

FIG. 33 is a perspective view of a matching lid for the base part of FIG. 32;

FIG. 34 is a perspective view of a closed package with a base part according to FIG. 32 and a lid according to FIG. 33;

FIG. 35 is an elevational view of a base and a lid having a base part with bars located only in a region extending between the front and rear lateral boundaries;

FIG. 36 is a top view of the base part of FIG. 35; and

FIG. 37 is a cross-sectional view through a portion of a base part and a lid showing a tongue and groove connection.

### DETAILED DESCRIPTION OF THE PREFERRED EMBODIMENTS

In the case 1 for artificial teeth 2 shown in the drawings, a base part 3 for receiving the teeth 2 and a lid 4 is provided that can be placed onto the base part 3 to cover it together with the inserted teeth 2. The boundaries 5, 6, and 7, 8 of the base part 3 and the lid 4 facing each other are embodied with different heights, that are adjusted to each other. Here, at least a large portion of the rear boundary 5 of the base part 3 has a height H1, which is embodied greater than the height H2 of the opposite front boundary 7 of the base part 3.

The rear and perhaps also the opposite front boundary 6 and/or 7 of the base part are provided with an edge 9, 10 extending in a convexly arched fashion and the respective edges 11 of the lid 4 are embodied indented corresponding thereto.

According to another embodiment, not shown, instead of an edge extending convexly arched, an edge extending concavely arched may also be provided at the base part 3. Here, the lower edge of the lid 4 is then adjusted appropriately.

As shown in FIGS. 28-30, another embodiment 1′ provides that the rear and perhaps also the opposite frontal boundary 5′, 7′ of the base part 3′ and the corresponding rear and frontal boundaries 6′, 8′ of the lid 4′ are provided with staggered, different height sections 25, 27; 28, respectively, following each other. An alternate arrangement of the staggered section 27′ on the front boundary 7″ for a base 3″ is shown in FIG. 28. The rear boundary can be similarly staggered. The corresponding lid (not shown) would have a complementary arrangement.

The upper edge 9, 10 of the rear and the front boundaries 5, 7 can also extend in a straight-line, as for example discernible from FIGS. 2-4 and 8. The edges at the lid 4 are then adjusted in the same manner, of course.

The lateral boundaries 12, 13, and 14, of the base part 3 and the lid 4 15 facing each other show a diagonally, perhaps arc-shaped extending edge 16, 17 adjusted to the different height rear and front boundaries 5, 7 and/or 6, 8 of the base part 3 and the lid 4.

As shown in FIGS. 32-34, in order for the base part 3 and the lid 4, after being placed thereupon, to be connected to each other relatively well protruding bars 29 may be arranged at the freely projecting rear and front boundaries 5, 7, and/or 6, 8 and perhaps also at the lateral boundaries 12, 13 and/or 14, 15 of the base part 3 and/or the lid 4 at the interior and/or the exterior sides of the boundaries for a mutually form-fitting engagement of the base part 3 and the lid 4, as shown in FIG. 34. Here, it is also possible to provide the protruding bars only over some sections of the circumference of the boundaries 5, 7 and/or 6, 8 or preferably only in the edge areas at the transfer of the rear and the front boundaries 5, 7 and/or 6, 8 to the lateral boundaries 12, 13, and/or 14, 15. This is illustrated in FIGS. 35 and 36, where the bars 29′ are located only at the lateral boundaries 12, 13.

Another design solution allows the base part 3 and the lid 4 to engage each other in a tongue-and-groove like fashion, as shown in FIG. 37, where a cross-section through an area of the interface between the base 3 and lid 4 is shown. This also represents the easiest solution with regard to construction.

In order for the inserted artificial teeth 2 to be held sufficiently tight in the case 1 and also be arranged appropriately for storage and transportation, a mass 18 for impressing artificial teeth 2 is provided in the base part, which is perhaps embodied elevated according to the arc-shaped or staggered edge in the central area in reference to the length of the base part 3.

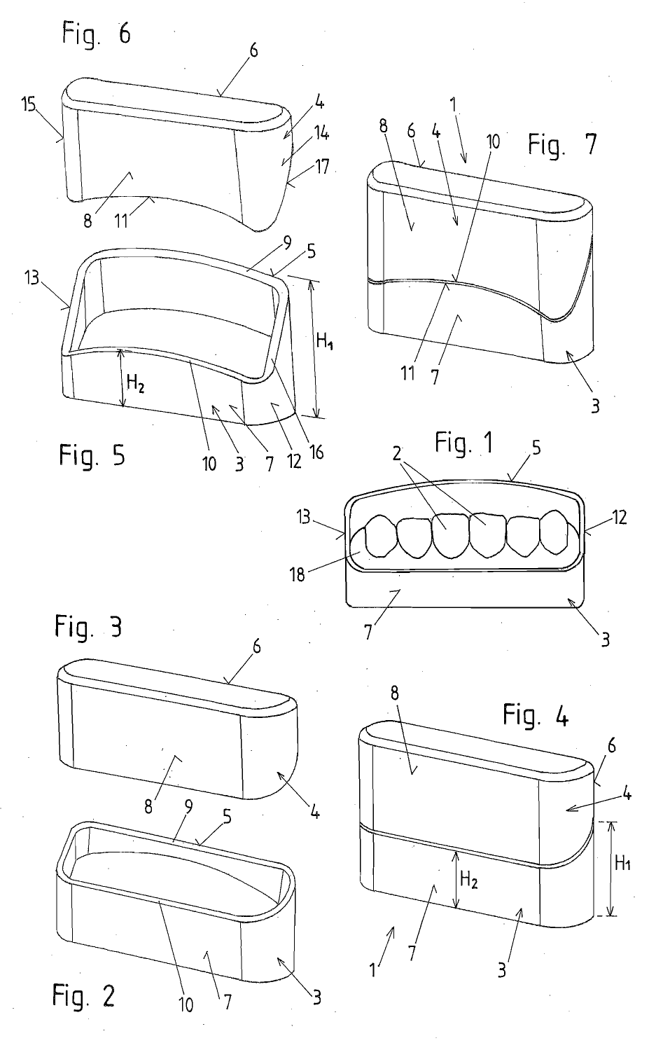

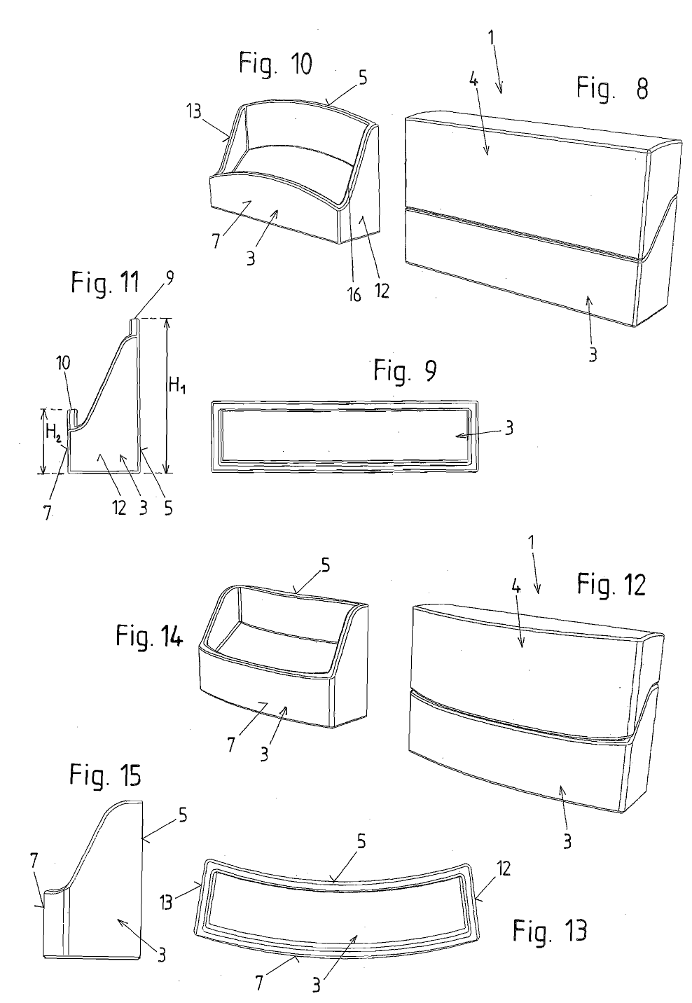

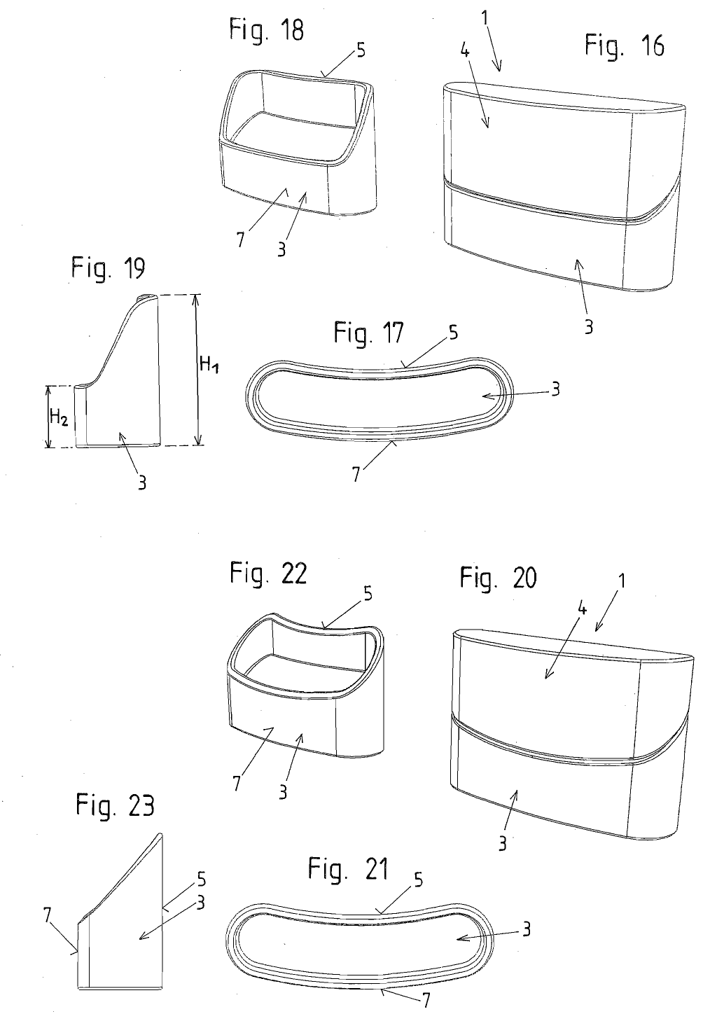

Within the scope of the invention, the case 1 can be provided with different exterior contours, which may also consider an easier storage and stacking. For example, it is possible to embody the case 1, as shown according to FIGS. 1 through 7, with front and rear plane surfaces parallel in reference to each other and lateral walls extending arc-shaped. However, it is also possible, as shown in FIGS. 8 through 11, to embody the base part 3 and the lid 4 such that the case 1 is embodied essentially rectangular, seen in a horizontal cross-section. According to an embodiment according to FIGS. 12 through 15, arc-shaped front and rear boundaries 5, 7 are provided extending parallel to each other and plane lateral boundaries 12, 13 and/or 14, 15. Here, the base part 3 and the lid 4 are provided with rear and front boundaries 5, 7, preferably curved in the same direction, seen in a horizontal cross-section, and perhaps plane or arc-shaped extending lateral boundaries positioned therebetween. This also applies equivalently for the embodiments according to FIGS. 16 through 19 as well as FIGS. 20 through 23.

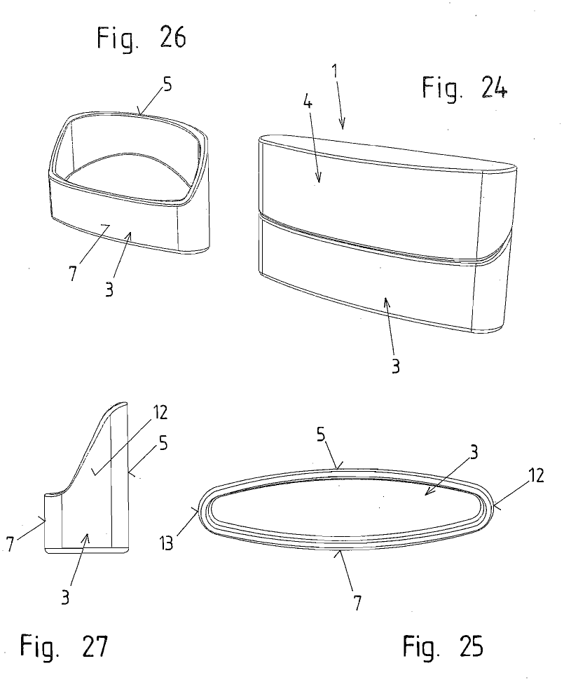

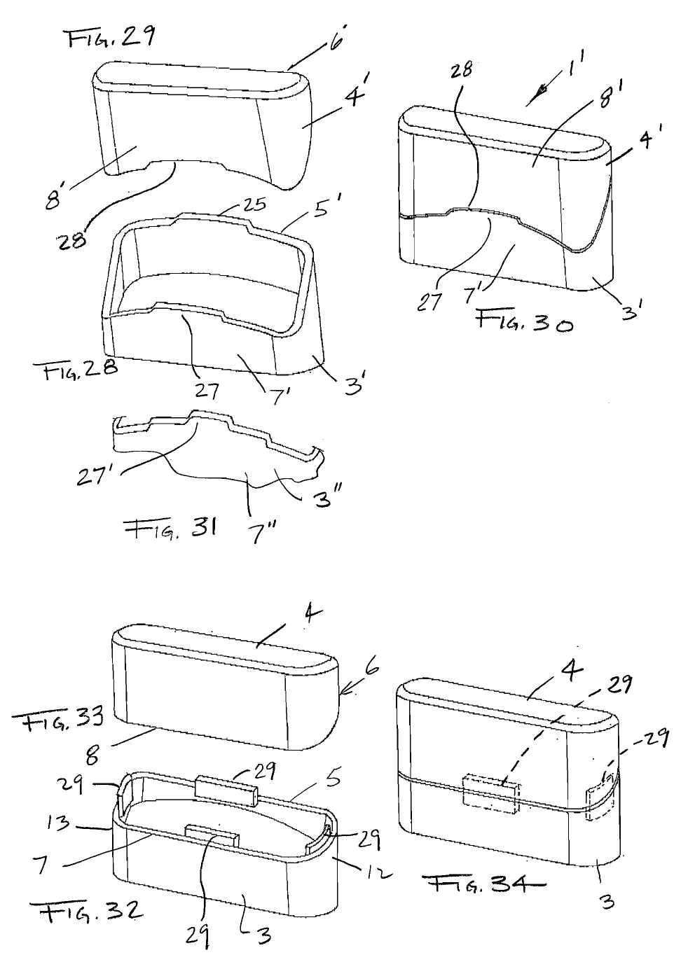

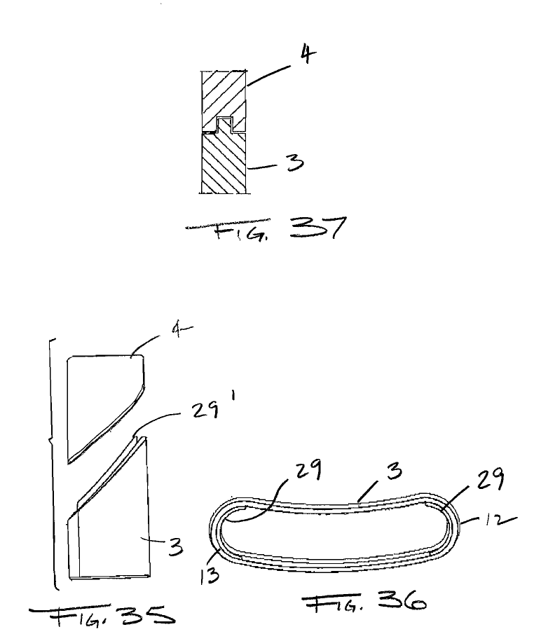

In the embodiment according to FIGS. 24 through 27 the case 1 is embodied such that the base part 3 and the lid 4 are embodied preferably ellipsoid, seen in a horizontal cross-section. Here, it is discernible that a multitude of options can be selected for the exterior design of the packaging. However, it is always essential and important that the rear boundary 5 of the base part 3 is embodied higher than the front boundary 7 of the base part 3. In an adjustment thereto the lid 4 is embodied correspondingly.

According to another embodiment it may also be provided that at least the lid 4 is made from a clear or at least translucent plastic. Then it can constantly be determined if any teeth 2 are stored in the case 1 at all, and perhaps also what type of teeth 2 are held there having what colors.

The case 1 and thus the base part 3 and the lid 4 are generally produced from a plastic material. It is therefore also possible to design the case 1 or the base part 3 and the lid 4 in the same or different colors. Further, the case 1 can be provided with the data of the inserted teeth at the base part 3 and/or the lid 4. Thus, labels may be adhered thereon. Such labels can be arranged overlapping the separation line between the base part 3 and the lid 4 in order to form a type of seal. The mass 18 for holding the teeth 2 in the base part can for example be molded or an elastic material.

As discernible from the above description the range of the invention is not limited to the exemplary embodiments shown, but should be determined to its full extent according to the attached claims as well as potential equivalent ones. While the above description and the drawings show the invention it is obvious for one trained in the art that different modifications can be made without leaving the true spirit and range of the invention.

1. A case for artificial teeth, comprising a base part for receiving the teeth and a lid that can be placed upon the base part that covers the base part together with the inserted teeth, with front and rear boundaries (7, 5) of the base part (3) having different heights and front and rear boundaries a (8, 6) of the lid (4) having different heights complementary to the base part, and at least a large part of the rear boundary (5) of the base part (3) is embodied higher than the front boundary (7) of the base part.

2. A case according to claim 1, wherein the rear boundary (5) of the base part (3) has an edge (9) extending convexly arched, and a corresponding edge of the lid (4) is indented correspondingly.

3. A case according to claim 2, wherein the front boundary (7) of the base part (3) has an edge extending convexly arched and the front boundary (8) of the lid (4) is indented correspondingly.

4. A case according to claim 1, wherein the rear boundaries of the base part (3) and the lid (4) have the different height sections that follow each other in a staggered fashion.

5. A case according to claim 4, wherein the front boundaries of the base part (3) and the lid (4) have the different height sections following each other in a staggered fashion.

6. A case according to claim 1, wherein mutually opposite lateral boundaries (12, 13 and/or 14, 15) of the base part (3) and the lid (4) have diagonally extending edges (16, 17) adjusted to the different height rear and front boundaries (5, 7 and/or 6, 8) of the base part (3) and the lid (4).

7. A case according to claim 6, wherein the mutually opposite lateral boundaries of the base part (3) and the lid (4) have an edge extending arc-shaped corresponding to the different height rear and front boundaries of the base part (3) and the lid (4).

8. A case according to claim 1, wherein protruding bars are arranged on at least one of the rear and front boundaries (5, 7) of the base part (3) or the lid (4) on at least one of an interior or exterior side of the boundaries (5, 7, 6, 8) for a mutual form-fitting engagement of the base part (3) and the lid (4).

9. A case according to claim 8, wherein the protruding bars are also arranged at lateral boundaries (12, 13 and/or 14, 15) of at least one of the base part (3) or the lid (4) on at least one of the interior or exterior sides of the boundaries for a mutual form-fitting engagement of the base part (3) and the lid (4).

10. A case according to claim 9, wherein the protruding bars are only provided over portions of a periphery of the boundaries (5, 7, 6, 8).

11. A case according to claim 10, wherein the protruding bars are only provided in corner sections at a transfer from the rear and front boundaries (5, 6, 7, 8) to the lateral boundaries (12, 13, 14, 15).

12. A package according to claim 1, wherein the base part (3) and the lid (4) engage each other in a tongue-and-groove fashion.

13. A package according to claim 2, further comprising a mass (18) located in the base part (3) to impress the artificial teeth (2).

14. A package according to claim 13, wherein the mass (18) includes an elevated central section in reference to a length of the base part (3) according to an arc-shaped or staggered edge.

15. A case according to claim 1, wherein the base part (3) and the lid (4) are essentially rectangular in a horizontal cross-section.

16. A case according to claim 1, wherein the base part (3) and the lid (4) are ellipsoid in a horizontal cross-section.

17. A case according to claim 1, wherein the base part (3) and the lid (4) include the front and rear boundaries (5, 7, 6, 8) curved in the same direction in a horizontal cross-section, with lateral boundaries (12, 13, 14, 15) located therebetween.

18. A case according to claim 1, wherein at least the lid (4) comprises a clear or at least translucent plastic.
